(19) United States
(12) Patent Application Publication
(10) Pub. No.: US 2015/0223940 A1
(43) Pub. Date: Aug. 13, 2015

(54) Extended Deltoid Tensioning Humeral Head

(71) Applicant: Anastasios Papadonikolakis, Fayetteville, NC (US)

(72) Inventor: Anastasios Papadonikolakis, Fayetteville, NC (US)

(21) Appl. No.: 14/463,914

(22) Filed: Aug. 20, 2014

Related U.S. Application Data
(60) Provisional application No. 61/939,455, filed on Feb. 13, 2014.

(51) Int. Cl. A61F 2/40

(52) U.S. Cl. CPC A61F 2/4003; A61F 2/4014; A61F 2002/4011; A61F 2002/4007; A61F 2002/4022

(57) ABSTRACT

An extended deltoid tensioning humeral head which includes a humeral prosthetic and a deltoid tensioning device to replace a shoulder joint for a patient that may be suffering from shoulder arthritis or poor rotator cuff functionality. The humeral prosthetic consists of a humeral head and a humeral stem to replace the humerus of the patient. The deltoid tensioning device provides a means to tension the muscle fibers of the deltoid providing increased abduction motion of the arm. The extended deltoid tensioning humeral head provides an anatomical alternative for the replacement of a shoulder joint opposed to the reverse total shoulder arthroplasty.

The current application claims a priority to the U.S. Provisional Patent application Ser. No. 69/939,455 filed on Feb. 13, 2014.

### FIELD OF THE INVENTION

The present invention relates generally to prosthetic implants. More specifically, the present invention relates to a shoulder or humeral deltoid tensioning implant. The purpose of this invention is to provide a humeral implant that increases the tension to the deltoid muscle.

### BACKGROUND OF THE INVENTION

Patients with shoulder arthritis that have torn rotator cuff muscle(s) have superior migration of the humerus and diminished tension on the deltoid muscle. In addition to the pain these patients have inability to raise their arm. A variety of humeral head implants exist for replacing humeral head surfaces. Conventional humeral head implants fail to accommodate patients having inadequate rotator cuff muscles and poor tension on the deltoid muscle. There is a need for humeral head implants that increase the tension to the deltoid muscle to allow for the range of motion and strength of the shoulder to be maintained. An existing shoulder implant that increases the tension of the deltoid muscle is the reverse total shoulder arthroplasty. This implant requires a ball and socket joint to be inserted into the patient's shoulder and upper arm. The ball portion of the joint is screwed into the scapula of the patient. A cup like attachment is inserted into the patient's upper arm to connect to the inserted ball portion, allowing the patient a full range of motion. This method of shoulder replacement, requires enough bone stock at the glenoid for fixation of the ball to the scapula.

The present invention provides a humeral implant that increases the tension to the deltoid muscle allowing the patient to retain the range of motion of their arm and the strength of the associated muscles without the need of insertion of an implant to the scapula. The present invention consists of a hemispherical or near hemispherical humeral head and an extended implant protruding from a portion of the humeral head. The extended implant displaces the deltoid muscle and increases the tension of its fibers.

### BRIEF DESCRIPTION OF THE DRAWINGS

FIG. 1 is a perspective illustration of the present invention.

FIG. 2 is a side profile of the present invention showing the connections of components.

FIG. 3 is another side profile of the present invention.

FIG. 4 is a perspective view of the humeral head of the present invention.

FIG. 5 is a side view of the humeral head of the present invention.

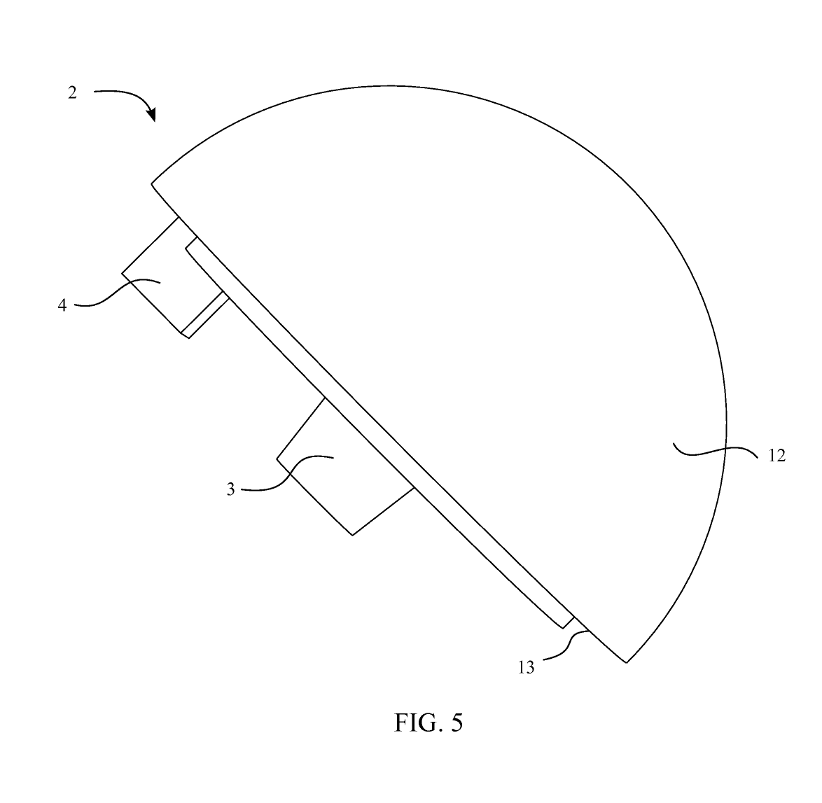

FIG. 6 is a side perspective of the deltoid tensioning device.

FIG. 7 is a front view of the inner concave surface of the deltoid tensioning device of the present invention.

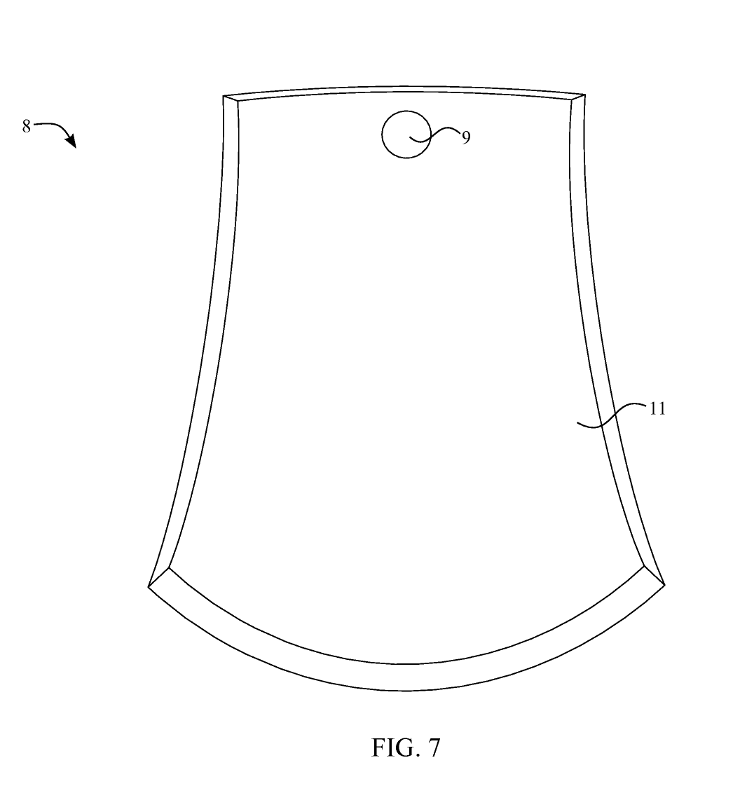

FIG. 8 is a perspective view of the humeral stem of the present invention.

FIG. 9 is a perspective view of an alternate embodiment of the present invention.

FIG. 10 is a side view of an alternate embodiment of the present invention.

FIG. 11 is a perspective view of the humeral head of the alternate embodiment of the present invention.

FIG. 12 is a perspective view of the deltoid tensioning device of an alternate embodiment of the present invention.

FIG. 13 is a front view of the inner concave surface of the deltoid tensioning device of an alternate embodiment of the present invention.

FIG. 14 is a side view of the humeral stem of an alternate embodiment of the present invention.

FIG. 15 is a perspective view of the humeral stem of an alternate embodiment of the present invention.

### DETAIL DESCRIPTIONS OF THE INVENTION

All illustrations of the drawings are for the purpose of describing selected versions of the present invention and are not intended to limit the scope of the present invention.

The present invention for an extended deltoid tensioning humeral head is a prosthetic implant which increases the ability for the patient to create tension in the deltoid muscle. While maintaining the normal anatomy of the humeral joint, the present invention will provide an alternative to existing shoulder replacements. The configuration of the present invention allows for an increase in the abduction moment arm and the stability of the shoulder joint. Patients having trouble with shoulder arthritis who are unable to raise their arm or suffer from poor rotator cuff muscle function should benefit from the present invention.

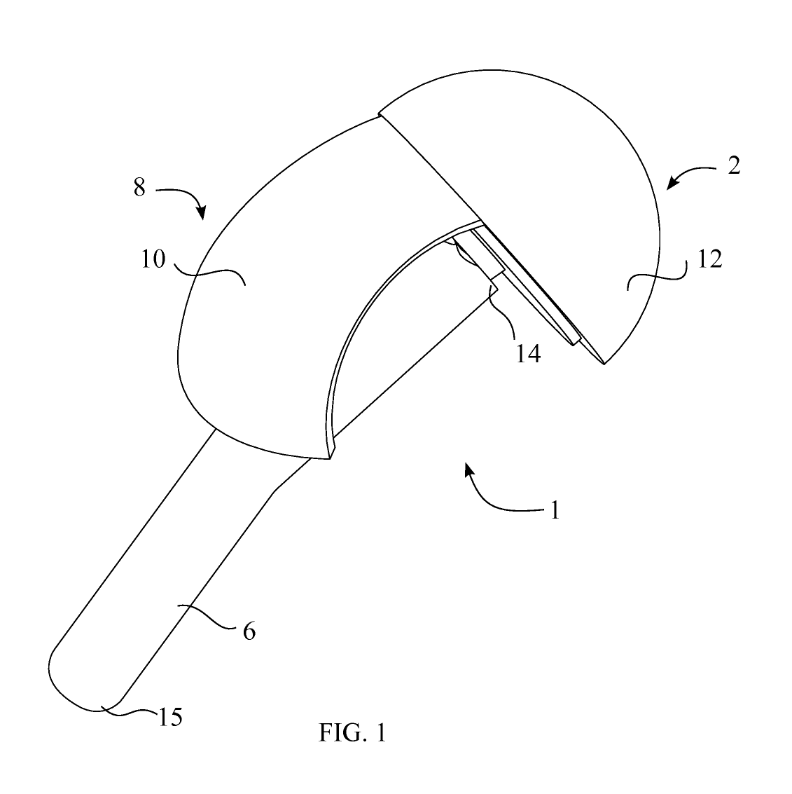

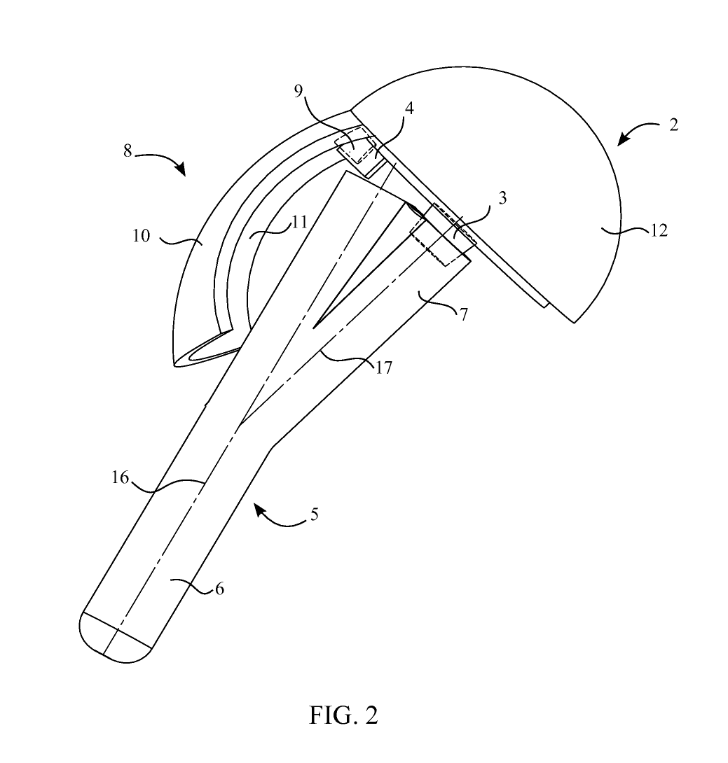

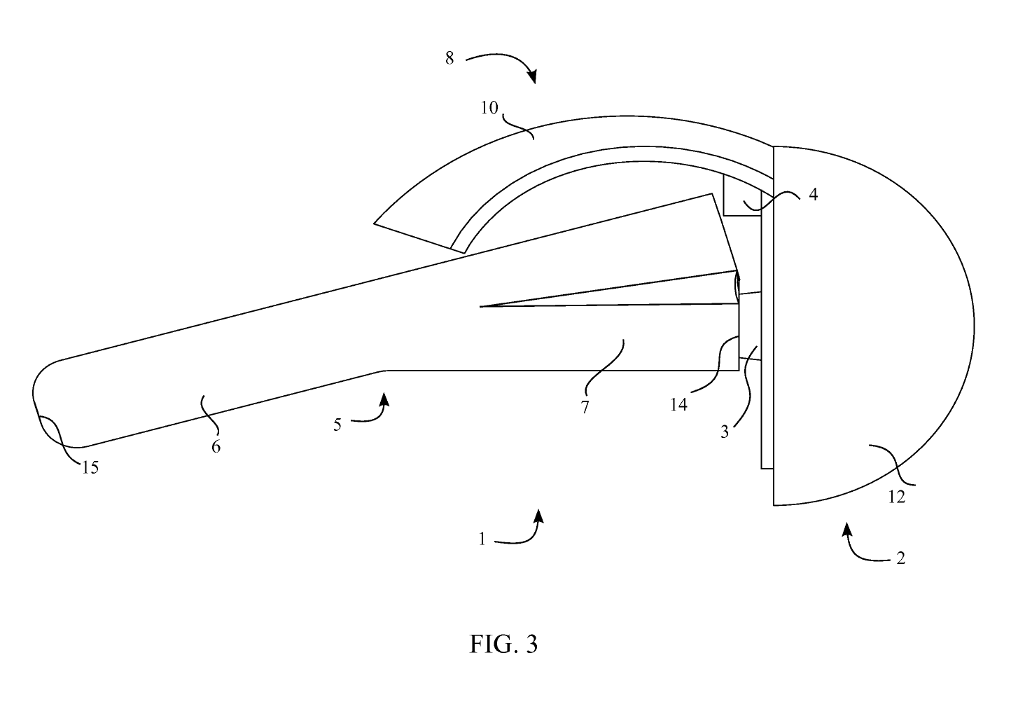

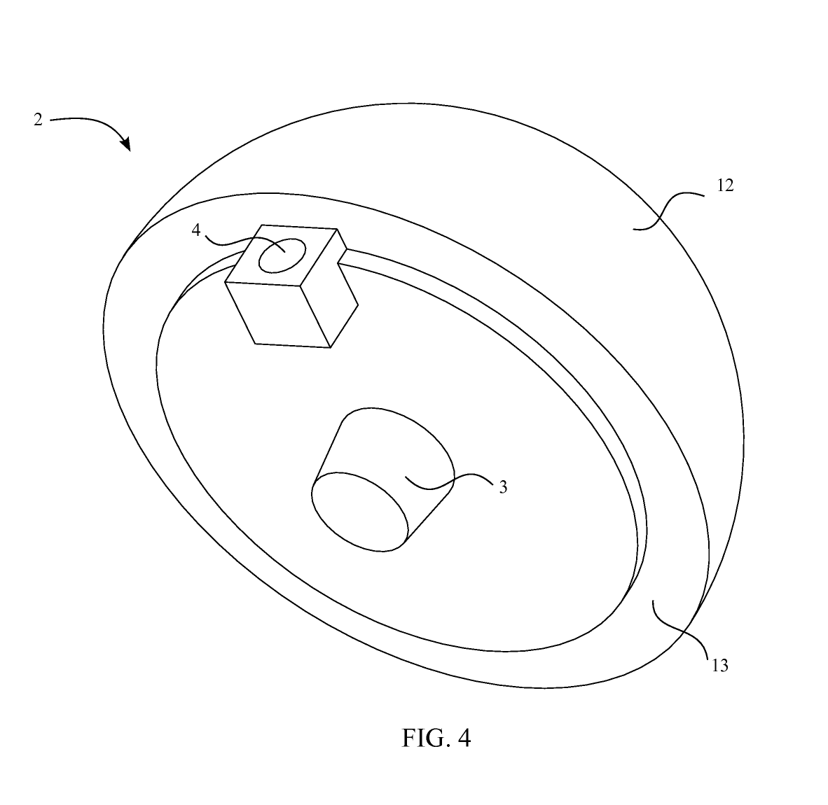

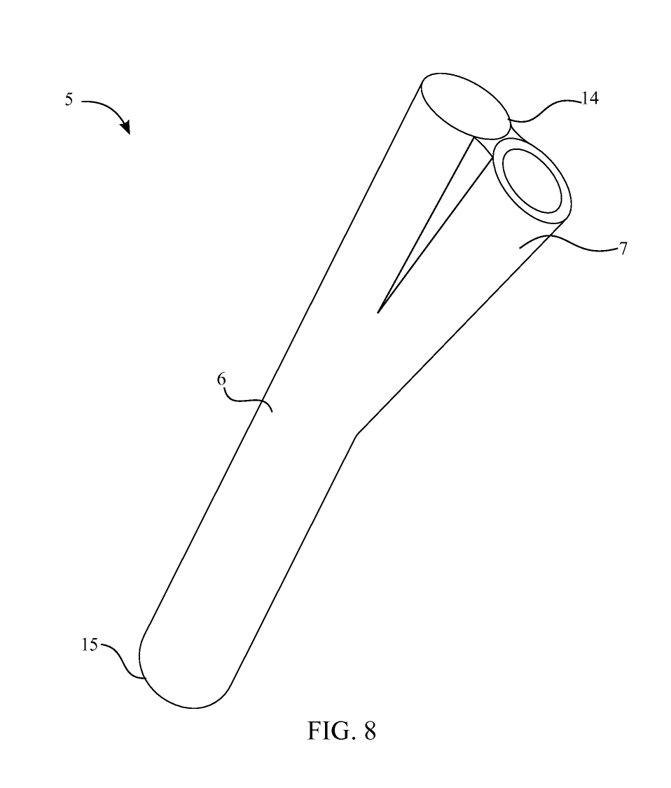

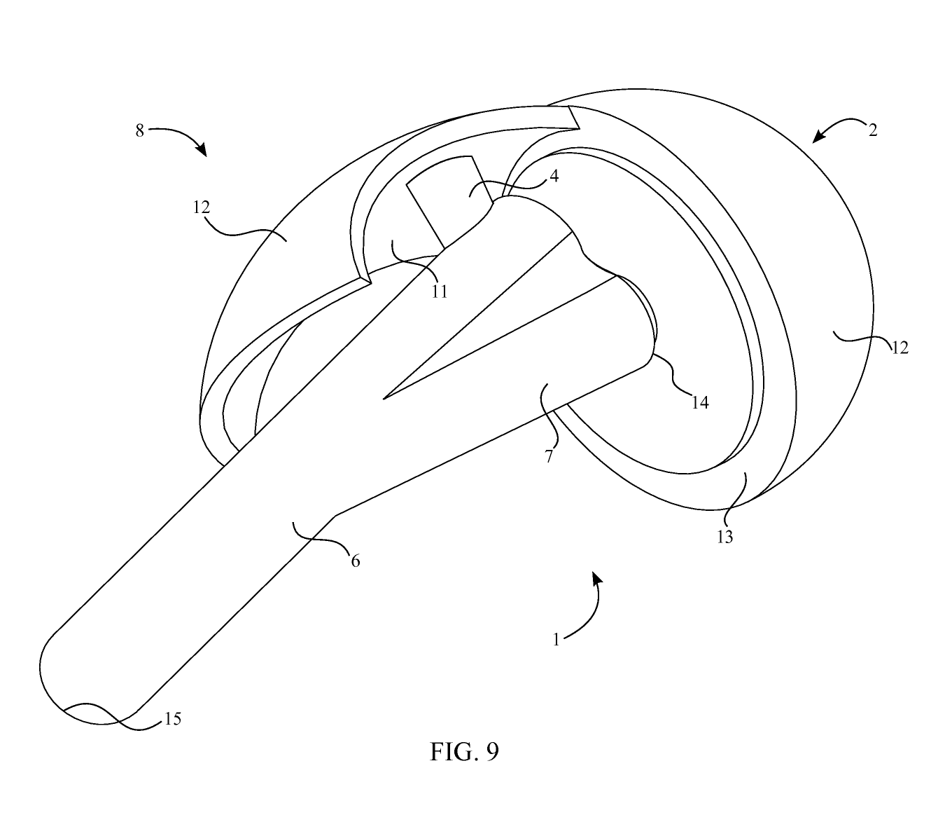

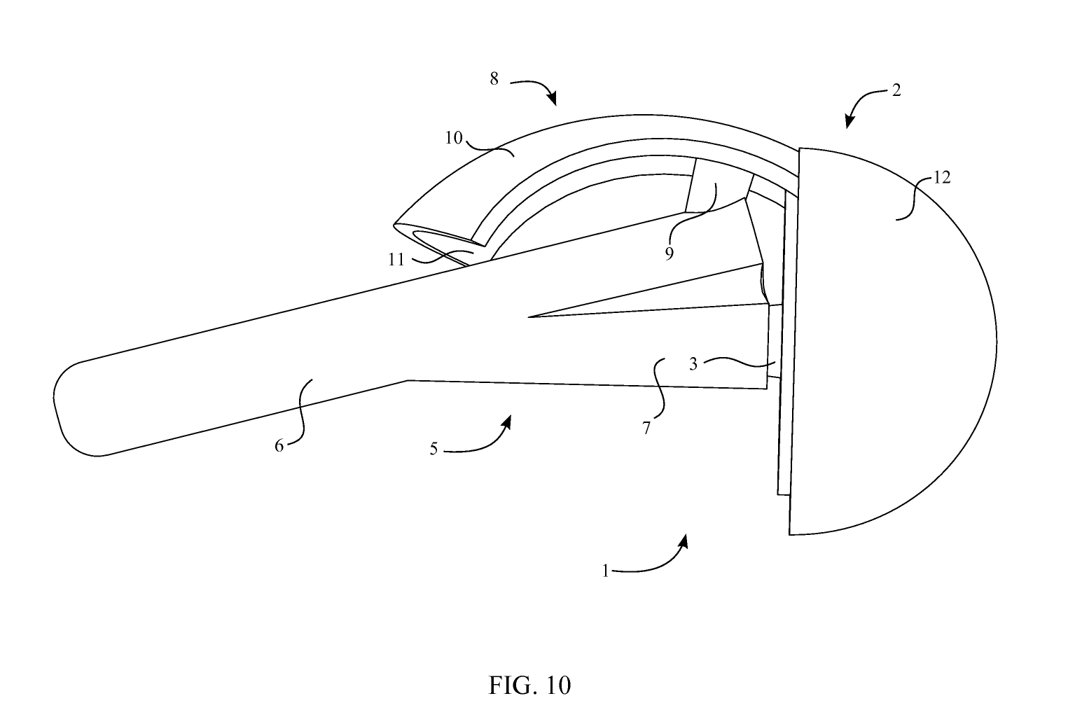

In accordance to FIG. 1, FIG. 2, FIG. 3, FIG. 9, and FIG. 10, the present invention comprises a humeral prosthetic 1 and a deltoid tensioning device 8. The humeral prosthetic 1 is able to replace the ball joint of a shoulder if the patient suffers from shoulder arthritis or a torn rotator cuff, while allowing the full range of motion of the shoulder and tensioning of the deltoid muscle. The humeral prosthetic 1 comprises a humeral head 2 and a humeral stem 5. Detailed in FIG. 4, the humeral head 2 comprises a ball connection portion 12 to articulate with the glenoid and the coracoacromial arch of the shoulder and a head anchor 3. The head anchor 3 is fixed to the humeral head 2 to the humeral stem 5 as shown in FIG. 2, FIG. 3, and FIG. 10. In the preferred embodiment of the present invention, the ball connection portion 12 is hemispherical or near hemispherical. The shape of the ball connection portion allows for the humeral head 2 to maintain the anatomical connection similar to that of the humerus to the scapula. The head anchor 3 is positioned concentrically with the ball connection portion 12 to provide a full range of motion when the prosthetic is implanted into the patient. With respect to FIG. 8, the humeral stem 5 comprises an elongated body 6 and a head anchor receiver 7. The head anchor receiver 7 is positioned to receive the humeral head 2. The elongated body 6 mounts to or replaces the humerus of the patient. The elongated body 6 comprises a proximal end 14 and a distal end 15. The head anchor 3 is connected adjacent and normal to a planar surface 13 of the ball connection portion 12. The head anchor receiver 7 is mounted along the elongated body 6 at the proximal end 14. The head anchor receiver 7 being fixed to the head anchor 3. A central axis of the elongated body 16 is anatomically angled with the central axis of the ball connection portion 17, as seen in FIG. 2, such that the ball connection portion 12 and the elongated body 6 are arranged similarly to the humerus head and the rest of the humerus. The deltoid tensioning device 8 comprises an anchor stem 9, a convex outer surface 10, and a concave inner surface 11. The deltoid tensioning device 8 is cantilevered on the humeral prosthetic 1, as shown in FIG. 3. The deltoid tensioning device 8 is offset form the elongated body 6 opposite the head anchor receiver 7 from the central axis of the elongated body 16, shown in FIG. 2, to more easily articulate tensioning of the deltoid muscle. The convex outer surface 10 articulates with the deltoid of the patient to increase tension of the muscle and the concavity compression phenomenon through the tensioning of the deltoid muscle fibers.

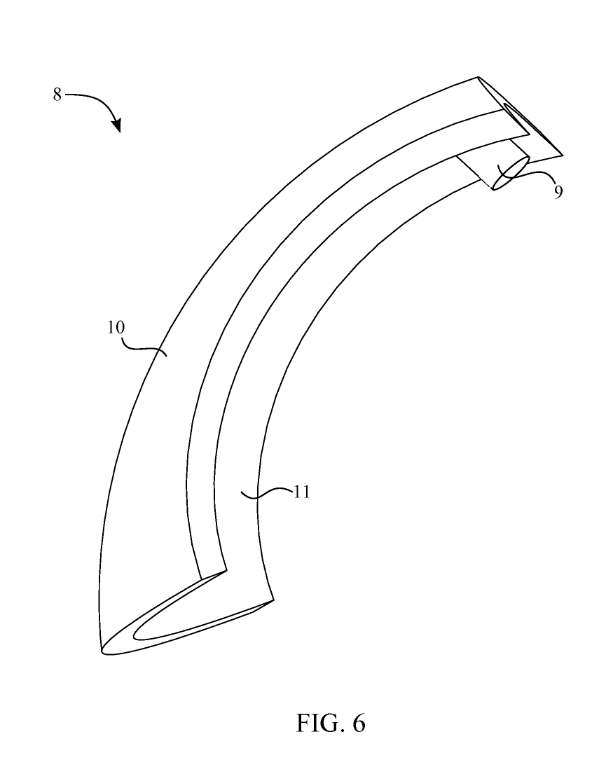

In reference to FIG. 6, the deltoid tensioning device has an anchor stem 9 connected adjacently to the concave inner surface 11. In the preferred embodiment, the humeral head 2 comprises an anchor stem receiver 4. The anchor stem 9 is fixed to the anchor stem receiver 4. The anchor stem receiver 4 is peripherally positioned on the planar surface 13. The anchor stem 9 is offset from the edge of the deltoid tensioning device 8. Thus, allowing the deltoid tensioning device 8 to be positioned adjacent to the planar surface 13 of the ball connection portion 12.

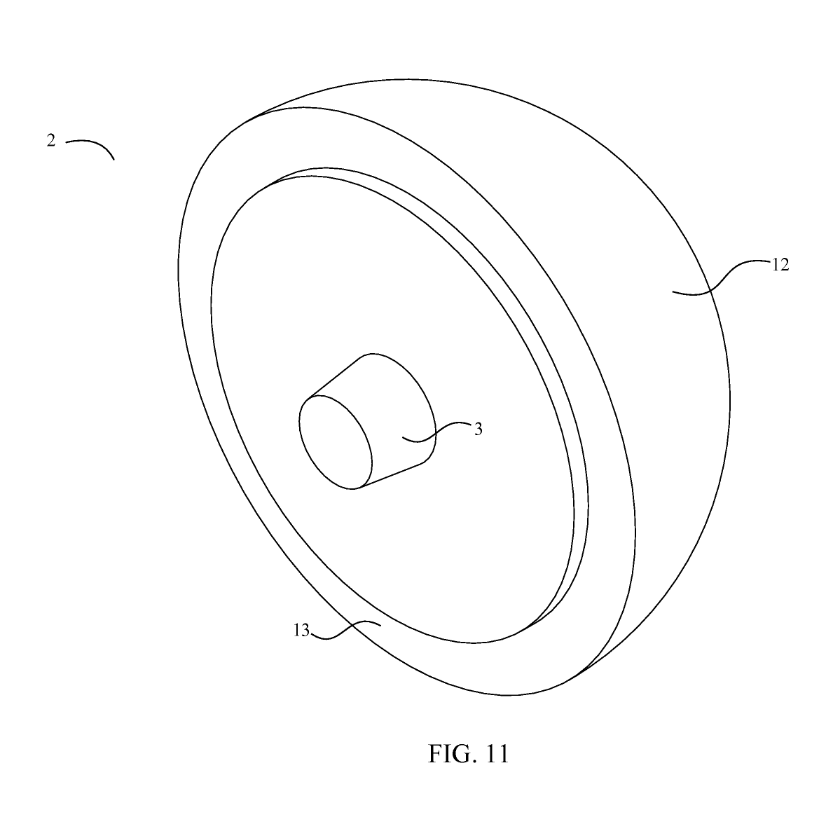

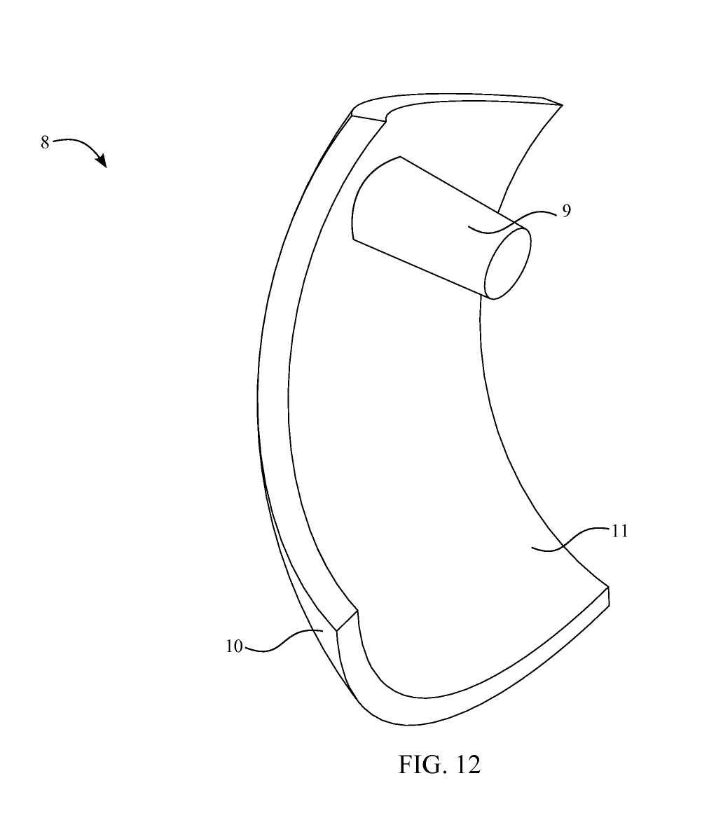

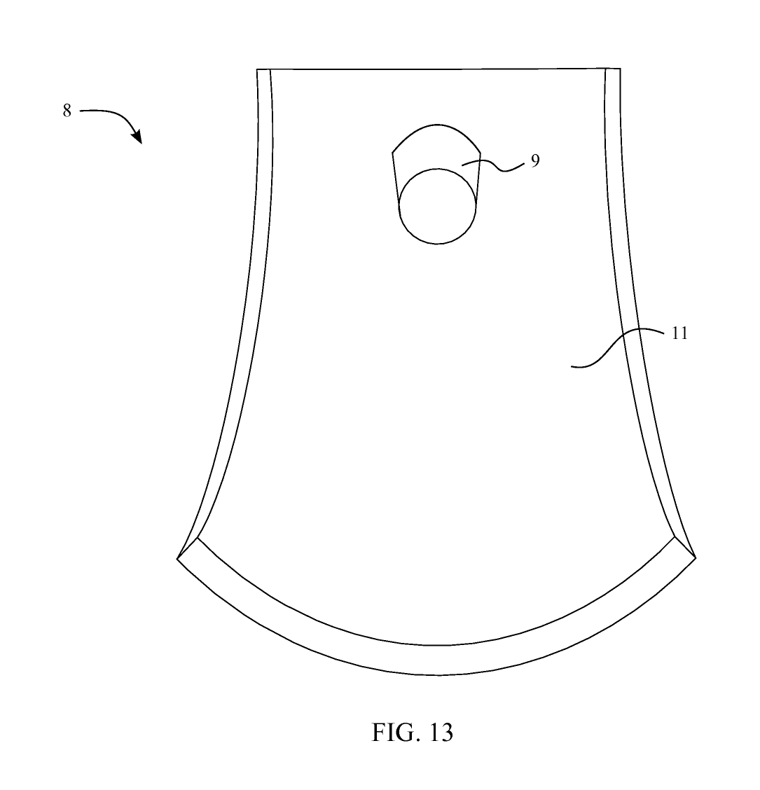

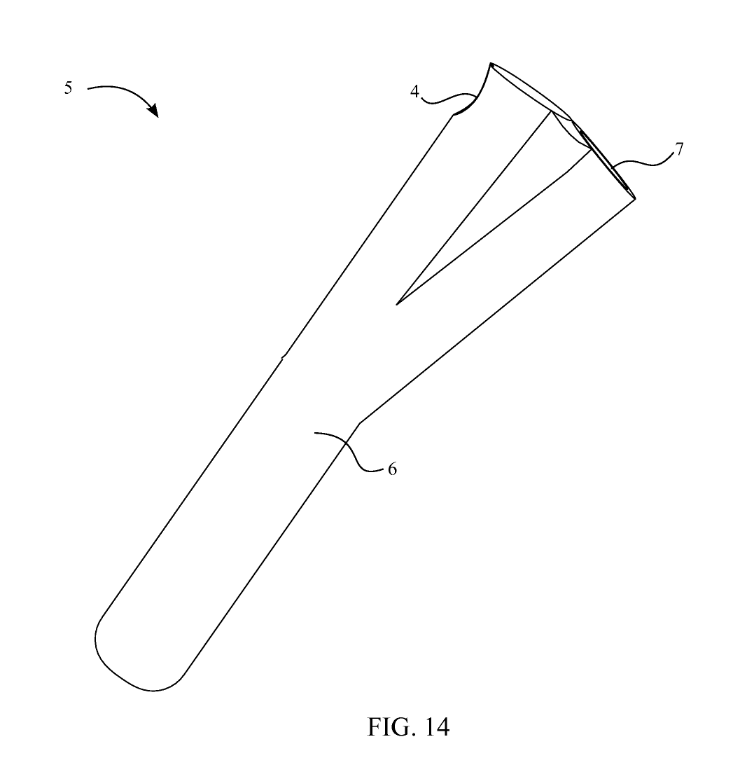

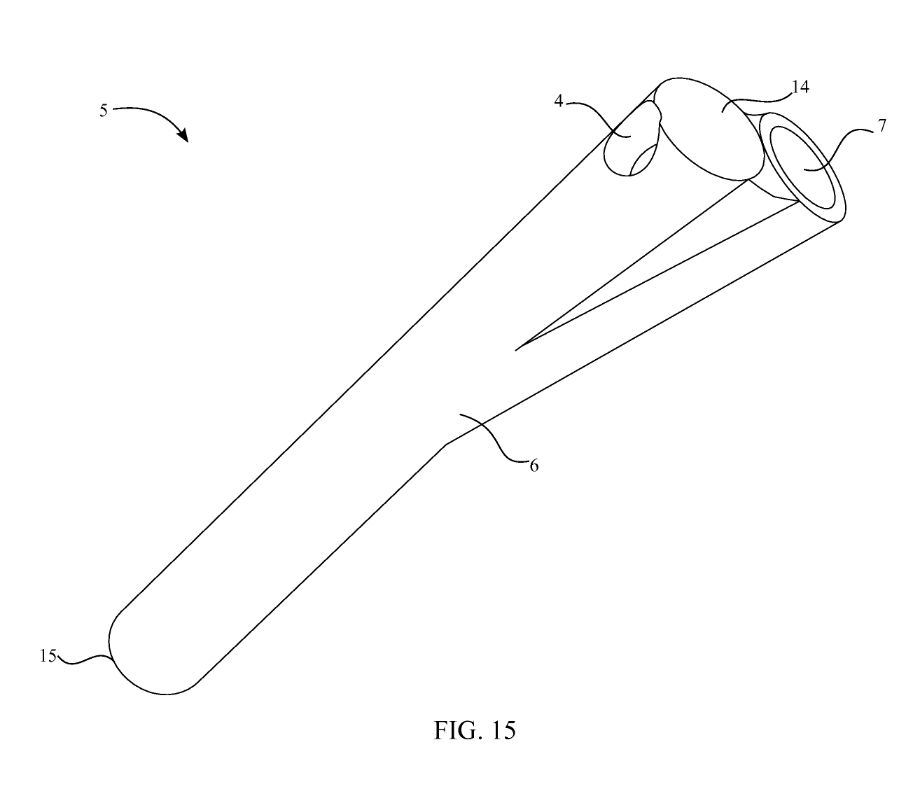

According to FIG. 9 to FIG. 15, in an alternate embodiment, the humeral stem 5 comprises the anchor stem receiver 4. The anchor stem receiver 4 is fixed to the anchor stem 9. For this embodiment, the anchor stem receiver 4 traverses into the elongated body 6 at the proximal end 14 of the humeral stem 5. The anchor stem receiver 4 is positioned adjacent and along the elongated body 6, opposite the head anchor receiver 7. The anchor stem 9 of this embodiment is generally larger than the anchor stem 9 of the preferred embodiment. The larger anchor stem 9 provides more strength to the connection, such that the anchor stem 9 stem is less likely to shear or break under stress due to the increase of cross-sectional area.

The head anchor 3 and the anchor stem 9 are preferred to be connected to the head anchor receiver 7 and the anchor stem receiver 4, respectively, through a morse taper connection. Although it is preferred, the connection is not limited to a morse taper, but may also include any connection means that will operatively connect the humeral head 2 to the humeral stem 5 and the deltoid tensioning device 8 to either the humeral head 2 or humeral stem 5 of the humeral prosthetic 1.

In the exemplary configuration of the present invention, the shoulder joint of the patient should increase the abduction moment arm of the deltoid muscle by increasing the distance between the center of rotation of the humeral head 2 and the muscle force vector that is generated by the deltoid muscle. The configuration should also decreases the superior migration of the humerus in the abducted position because the acrominon displaces the humeral head 2 inferiorly when the convex outer surface 10 of the deltoid tensioning device 8 comes in contact with the acromion.

Although the invention has been explained in relation to its preferred embodiment, it is to be understood that many other possible modifications and variations can be made without departing from the spirit and scope of the invention as hereinafter claimed.

What is claimed is:

1. An extended deltoid tensioning humeral head comprises:
a humeral prosthetic;
a deltoid tensioning device;
the humeral prosthetic comprises a humeral head and a humeral stem;
the humeral head comprises a ball connection portion and a head anchor;
the humeral stem comprises an elongated body and a head anchor receiver;
the elongated body comprises a proximal end and a distal end;
the deltoid tensioning device comprises an anchor stem, a convex outer surface, and a concave inner surface;
the head anchor being connected adjacent and normal to a planar surface of the ball connection portion;
the head anchor receiver being mounted along the elongated body at the proximal end;
the head anchor receiver being fixed to the head anchor;
a central axis of the elongated body being at an anatomical angle with a central axis of the ball connection portion;
the deltoid tensioning device being cantilevered on the humeral prosthetic; and
the deltoid tensioning device being offset from the elongated body opposite the head anchor receiver.

2. The extended deltoid tensioning humeral head as claimed in claim 1 comprises:
the ball connection portion being a hemispherical shape.

3. The extended deltoid tensioning humeral head as claimed in claim 1 comprises:
the head anchor being positioned concentrically with the ball connection portion.

4. The extended deltoid tensioning humeral head as claimed in claim 1 comprises:
the anchor stem being connected adjacent the concave inner surface.

5. The extended deltoid tensioning humeral head as claimed in claim 1 comprises:
the humeral head comprises an anchor stem receiver; and
the anchor stem receiver being fixed to the anchor stem.

6. The extended deltoid tensioning humeral head as claimed in claim 5 comprises:
the anchor stem receiver being peripherally positioned on the planar surface of the ball connection portion; and
the deltoid tensioning device being positioned adjacent to the planar surface of the ball connection portion.

7. The extended deltoid tensioning humeral head as claimed in claim 1 comprises:
the humeral stem comprises an anchor stem receiver; and
the anchor stem receiver being fixed to the anchor stem.

8. The extended deltoid tensioning humeral head as claimed in claim 7 comprises:
the anchor stem receiver traversing into the elongated body at the proximal end; and
the anchor stem receiver being positioned adjacent and along the elongated body, opposite head anchor receiver.

9. An extended deltoid tensioning humeral head comprises:
a humeral prosthetic;
a deltoid tensioning device;
the humeral prosthetic comprises a humeral head and a humeral stem;
the humeral head comprises a ball connection portion and a head anchor;
the humeral stem comprises an elongated body and a head anchor receiver;
the elongated body comprises a proximal end and a distal end;
the deltoid tensioning device comprises an anchor stem, a convex outer surface, and a concave inner surface;
the head anchor being connected adjacent and normal to a planar surface of the ball connection portion;
the head anchor receiver being mounted along the elongated body at the proximal end;
the head anchor receiver being fixed to the head anchor;
a central axis of the elongated body being at an anatomical angle with a central axis of the ball connection portion;
the deltoid tensioning device being cantilevered on the humeral prosthetic;
the deltoid tensioning device being offset from the elongated body opposite the head anchor receiver; and
the head anchor being positioned concentrically with the ball connection portion.

10. The extended deltoid tensioning humeral head as claimed in claim 9 comprises:
the ball connection portion being a hemispherical shape.

11. The extended deltoid tensioning humeral head as claimed in claim 9 comprises:
the anchor stem being connected adjacent the concave inner surface.

12. The extended deltoid tensioning humeral head as claimed in claim 9 comprises:
the humeral head comprises an anchor stem receiver; and
the anchor stem receiver being fixed to the anchor stem.

13. The extended deltoid tensioning humeral head as claimed in claim 12 comprises:
the anchor stem receiver being peripherally positioned on the planar surface of the ball connection portion; and
the deltoid tensioning device being positioned adjacent to the planar surface of the ball connection portion.

14. The extended deltoid tensioning humeral head as claimed in claim 9 comprises:
the humeral stem comprises an anchor stem receiver; and
the anchor stem receiver being fixed to the anchor stem.

15. The extended deltoid tensioning humeral head as claimed in claim 14 comprises:
the anchor stem receiver traversing into the elongated body at the proximal end; and
the anchor stem receiver being positioned adjacent and along the elongated body, opposite head anchor receiver.

16. An extended deltoid tensioning humeral head comprises:
a humeral prosthetic;
a deltoid tensioning device;
the humeral prosthetic comprises a humeral head and a humeral stem;
the humeral head comprises a ball connection portion and a head anchor;
the humeral stem comprises an elongated body and a head anchor receiver;
the elongated body comprises a proximal end and a distal end;
the deltoid tensioning device comprises an anchor stem, a convex outer surface, and a concave inner surface;
the head anchor being connected adjacent and normal to a planar surface of the ball connection portion;
the head anchor receiver being mounted along the elongated body at the proximal end;
the head anchor receiver being fixed to the head anchor;
a central axis of the elongated body being at an anatomical angle with a central axis of the ball connection portion;
the deltoid tensioning device being cantilevered on the humeral prosthetic;
the deltoid tensioning device being offset from the elongated body opposite the head anchor receiver;
the head anchor being positioned concentrically with the ball connection portion;
the anchor stem being connected adjacent the concave inner surface; and
the ball connection portion being a hemispherical shape.

17. The extended deltoid tensioning humeral head as claimed in claim 16 comprises:
the humeral head comprises an anchor stem receiver; and
the anchor stem receiver being fixed to the anchor stem.

18. The extended deltoid tensioning humeral head as claimed in claim 17 comprises:
the anchor stem receiver being peripherally positioned on the planar surface of the ball connection portion; and
the deltoid tensioning device being positioned adjacent to the planar surface of the ball connection portion.

19. The extended deltoid tensioning humeral head as claimed in claim 16 comprises:
the humeral stem comprises an anchor stem receiver; and
the anchor stem receiver being fixed to the anchor stem.

20. The extended deltoid tensioning humeral head as claimed in claim 19 comprises:
the anchor stem receiver traversing into the elongated body at the proximal end; and
the anchor stem receiver being positioned adjacent and along the elongated body, opposite head anchor receiver.
